(19) United States
(12) Patent Application Publication
(10) Pub. No.: US 2010/0179422 A1
(43) Pub. Date: Jul. 15, 2010

(54) Dual Modality Imaging Of Tissue Using A Radionuclide

(76) Inventor: Michael K. O'Connor, Rochester, MN (US)

Correspondence Address: QUARLES & BRADY LLP, 411 E. WISCONSIN AVENUE, SUITE 2040, MILWAUKEE, WI, 53202-4497, US

(21) Appl. No.: 12/688,638

(22) Filed: Jan. 15, 2010

Related U.S. Application Data
(60) Provisional application No. 61/145,053, filed on Jan. 15, 2009.

(51) Int. Cl. A61B 6/00

(52) U.S. Cl. 600/431

(57) ABSTRACT

A method for imaging a subject includes injecting the subject with a single dose of a radionuclide and acquiring, with a molecular breast imaging (MBI) system, a first set of medical image data of a breast of the subject after injection of the single dose of a radionuclide. The method also includes acquiring, with a myocardial perfusion imaging (MPI) system, a second set of medical image data of a portion of a cardiovascular system of the subject after the single dose of a radionuclide and reconstructing the first set of medical image data into a medical image of the breast of the subject and reconstructing the second set of medical image data into a medical image of the portion of the cardiovascular system of the subject.

### REFERENCE TO RELATED APPLICATIONS

This application is based on provisional application Ser. No. 61/145,053, filed Jan. 15, 2009, and entitled “DUAL MODALITY IMAGING OF TISSUE AFTER ADMINISTRATION OF A RADIONUCLIDE,” and claims the benefit thereof.

### FIELD OF THE INVENTION

The present invention related to systems and methods for imaging of organic tissue, and more particularly, to a system and method for using a radionuclide as an agent imaging, such as in imaging breast and myocardial tissue.

### BACKGROUND OF THE INVENTION

Cardiovascular disease and cancer remain the leading causes of death in America. In fact, women suffer a higher risk of mortality resulting from cardiovascular disease than from cancer. In 2007 alone, almost twice as many women died of cardiovascular disease (both heart and stroke) than from all cancers combined. For example, studies have shown that one in three American women will die of heart disease, while approximately one in seven American women will get breast cancer during her lifetime. Consequently, many women undergo diagnostic imaging, including nuclear imaging, procedures in order to test for such diseases.

Cardiovascular Imaging

Cardiovascular disease may be diagnosed through the use various cardiology imaging procedures, such coronary angiography, computed tomography imaging, magnetic resonance imaging (MRI), position emission tomography (PET) imaging, or myocardial perfusion imaging (MPI).

The most common type of nuclear cardiology imaging procedure is MPI, in which myocardial blood flow is evaluated in order to diagnose myocardial scarring or ischemia, for example. MPI assesses heart function based on the absorption of an intravenously administered radionuclide, such as a radiopharmaceutical or radioactive tracer, in the myocardium. The myocardium regions extract and retain the administered radionuclide depending on the blood flow to the region. Images of the myocardium depict zones of diminished radionuclide concentration in the areas of decreased perfusion. Therefore, MPI can help identify areas of reduced blood flow that may be associated with cardiovascular disease.

The distribution of blood flow can be assessed at rest, at stress, or both. Commonly, MPI is performed at a rest condition and at a stress condition. A radionuclide, such as Technetium 99m, can be administered twice, once at rest, and again following cardiac stress that may be caused by exercise and/or pharmacologic stimulation. The myocardium is imaged at each condition using a gamma camera that produces scintigrams depicting coronary blood flow in the myocardium. The images taken at rest can be compared to the images taken at stress to assess perfusion abnormalities. For example, if the diminished radionuclide concentration is worse when the radionuclide is administered during stress as compared to when the radionuclide is administered during rest, the zone of decreased concentration during stress is likely due to ischemia. On the other hand, if the radionuclide concentration remains the same after an injection of the radionuclide at rest, the zone of decreased concentration is likely due to scar tissue.

Breast Cancer Imaging

Screening mammography has been the gold standard for breast cancer detection for over 30 years, and is the only available screening method proven to reduce breast cancer mortality. However, the sensitivity of screening mammography varies considerably. One factor in the failure of mammography to detect breast cancer is radiographic breast density. In studies examining the sensitivity of mammography as a function of breast density, it has been determined that the sensitivity of mammography falls from 87-97 percent in women with fatty breasts and to 48-63 percent in women with extremely dense breasts.

Diagnostic alternatives to mammography include ultrasound and MRI. The effectiveness of whole-breast ultrasound as a screening technique does not appear to be significantly different from mammography. MRI has a high sensitivity for the detection for breast cancer and is not affected by breast density. However, since bilateral breast MRI is currently approximately 20 times more expensive than mammography, it is not in widespread use as a screening technique.

Another technology is positron emission mammography (PEM). This uses two, small, opposing PET detectors to image the breast. The PEM technology offers excellent resolution. However, the currently available radiotracer, F-18 Fluoro deoxyglucose, requires that a patient fast overnight; the patient have low blood levels (this is often a problem for diabetics); and the patient should wait 1-2 hours after the injection for optimum uptake of F-18FDG in the tumor. The high cost of these PET procedures coupled with the long patient preparation time reduces the usefulness of this procedure and makes it difficult to employ for routine breast evaluation.

Radionuclide imaging of the breast (scintimammography) with Tc-99m sestamibi was developed in the 1990s and has been the subject of considerable investigation over the last 10-15 years. This functional method is not dependent upon breast density. Large multi-center studies have shown the sensitivity and specificity of scintimammography in the detection of malignant breast tumors to be approximately 85 percent. However, these results only hold for large tumors and several studies have shown that the sensitivity falls significantly with tumor size. The reported sensitivity for lesions less than 10-15 mm in size was approximately 50 percent. This limitation is particularly relevant in light of the finding that up to a third of breast cancers detected by screening mammography are smaller than 10 mm. Prognosis depends on early detection of the primary tumor. Spread of a cancer beyond the primary site occurs in approximately 20-30 percent of tumors 15 mm or less in size. However, as tumor size grows beyond 15 mm, there is an increasing incidence of node positive disease, with approximately 40 percent of patients having positive nodes for breast tumors 2 cm in diameter. Hence, for a nuclear medicine technique to be of value in the primary diagnosis of breast cancer, it should be able to reliably detect tumors that are less than 15 mm in diameter. The failure of conventional scintimammography to meet this limit led to its abandonment as a useful technique in the United States.

In an attempt to overcome the limitation of conventional scintimammography, several small field-of-view gamma cameras have been developed that permit the breast to be imaging in a similar manner and orientation to conventional mammography. One commercial system for single photon imaging that is currently available is that manufactured by Dilon Technologies of Newport News, VA. Using a small detector and compression paddle, they reported a sensitivity of 67 percent for the detection of sub-10 mm lesions.

Therefore, various imaging modalities utilize radionuclides, such as Tc-99m sestamibi or Tc-99m tetrofosmin, to image organic tissue. The dosage of the radionuclide utilized may depend on the organ being studies. When administered in small amounts, the radiation the body receives is low. However, when larger amounts are given, it may cause adverse effects on the body may ensue.

Therefore, it would be advantageous to provide a system and method diagnostic imaging that would reduce the amount of radiation exposed to the patient, while having adequate imaging resolution and not falling prey to the limitations of the systems and methods described above.

### SUMMARY OF THE INVENTION

The present invention overcomes the aforementioned drawbacks by providing a system and method for performing quantitative tumor analysis using information acquired with a dual-headed, high resolution molecular breast imaging (MBI) system at a regiment that can be coordinated with other nuclear imaging, such as with myocardial perfusion imaging. The MBI process can accommodate for an injection dosage that is higher than 20 mCi Tc-99m sestamibi or Tc-99m tetrofosmin, such as the dosages used during MPI. By recognizing that MBI is not impacted by uptake of these radiopharmaceuticals in other organs, such as the myocardium or the liver, the present invention permits breast imaging to commence shortly after the injection of the radiopharmaceutical, such as 5 minutes post-injection, and thereby, temporally couple an MBI procedure with other nuclear imaging processes to perform a breast analysis without the patient being subjected to any additional radiation dose.

In one implementation, both myocardial and breast tissue are imaged within temporal proximity of one another while the tissues uptake a previously administered radionuclide. Specifically, the patient is consecutively imaged using two different imaging modalities including a dual-headed molecular breast imaging (MBI) system. Alternatively, the breast tissue may be imaged both before and after a myocardial stress test of a myocardial perfusion scan in order to obtain diagnostic information as to the behavior of abnormal regions on the breast tissue images.

In accordance with one aspect of the invention, a method for imaging a subject is disclosed that includes injecting the subject with a single dose of a radionuclide and acquiring, with a molecular breast imaging (MBI) system, a first set of medical image data of a breast of the subject after injection of the single dose of a radionuclide. Contemporaneously therewith, the method includes acquiring, with a myocardial perfusion imaging (MPI) system, a second set of medical image data of a portion of a cardiovascular system of the subject after the single dose of a radionuclide. Furthermore, the method includes reconstructing the first set of medical image data into a medical image of the breast of the subject and reconstructing the second set of medical image data into a medical image of the portion of the cardiovascular system of the subject.

Various other features of the present invention will be made apparent from the following detailed description and the drawings.

### BRIEF DESCRIPTION OF THE DRAWINGS

Implementations will become more apparent from the detailed description set forth below when taken in conjunction with the drawings, in which like elements bear like reference numerals.

FIG. 1 is an illustration of a nuclear medicine-based, high-resolution, molecular breast imaging (MBI) system;

FIG. 2 is a flow chart setting forth the steps for determining a tumor size using the system of FIG. 1;

FIG. 3 is a schematic representation of a user interface for determining tumor size;

FIG. 4 is a flow chart setting for the steps for determining tumor depth and relative radiotracer uptake; and

FIG. 5 is a flow chart outlining an exemplary process for imaging of myocardial and breast tissue.

### DETAILED DESCRIPTION OF THE INVENTION

In nuclear medicine, both breast cancer and myocardial diseases can be connected through the fortuitous biological behavior of radiopharmaceuticals such as Tc-99mm sestabmibi and Tc-99m tetrofosimin. These radiopharmaceuticals were originally developed in the 1980s for myocardial perfusion imaging and have become the standard radiopharmaceuticals used in the United States in the evaluation of myocardial function. In the late 1980′s it was noticed that these compounds also localized in a variety of cancers and showed strong uptake in breast cancer. This finding lead to the development of a technique known as scintimammography. However, technical limitations significantly reduced the sensitivity of the technique for the detection of small breast tumors (sensitivity>50% for sub 10 mm lesions), and the technology never gained widespread clinical use. Recent studies have overcome some of these limitations. For example, a co-pending patent application with an Application Ser. No. WO/2008/073897, filed Dec. 10, 2007, entitled “System And Method For Quantitative Molecular Breast Imaging” discloses systems, apparatus, and methods for performing quantitative tumor analysis using ultra high-resolution, detectors configured in a molecular breast imaging (MBI) system. MBI has a high sensitivity (e.g., 93%) for the detection of lesions ranging in 5-10 mm in size, for example.

Molecular Breast Imaging (MBI)

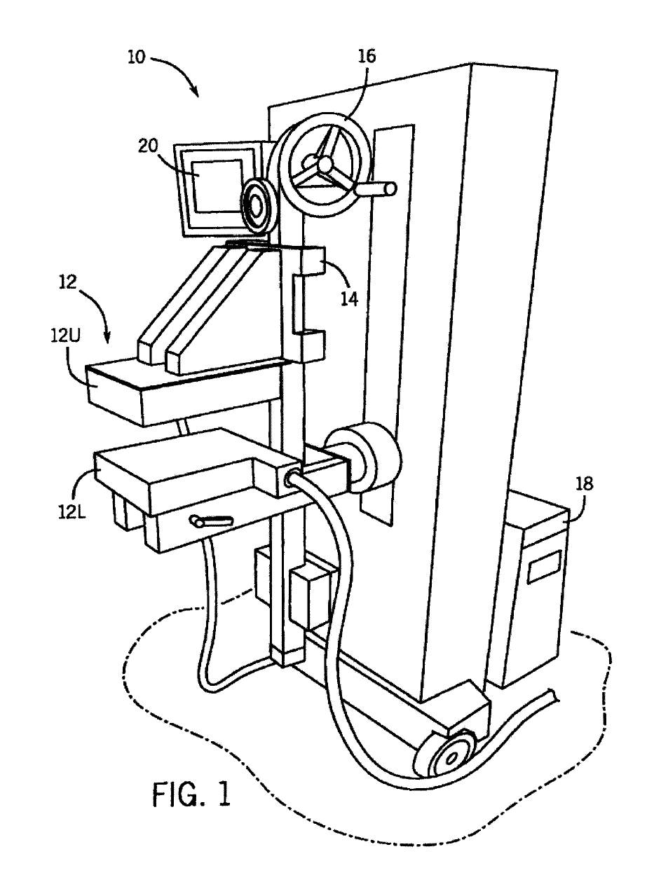

Referring to FIG. 1, a nuclear medicine-based, high-resolution molecular breast imaging (MBI) system 10 includes two opposing cadmium zinc telluride (CZT) detectors (detector heads) 12. In particular, the detector heads 12 include an upper detector head 12U and a lower detector head 12L. Each detector head 12U, 12L is, for example, 20 cm by 16 cm in size and mounted on a modified upright type mammographic gantry 14. In accordance with one embodiment, the detector heads 12 are LumaGEM 3200S high-performance, solid-state cameras from Gamma Medica having a pixel size of 1.6 mm. LumaGEM is a trademark of Gamma Medica, Inc. Corporation of California.

The relative position of the detector heads 12 can be adjusted using a user control 16. Specifically, the detector head assemblies 12 are, preferably, designed to serve as a compression mechanism. Accordingly, this system configuration reduces the maximum distance between any lesion in the breast and either detector head 12 to one-half of the total breast thickness, potentially increasing detection of small lesions without additional imaging time or dose. The MBI system 10 includes a processor 18 for processing the signals acquired by the detector heads 12 to produce an image, which may be displayed on an associated display 20.

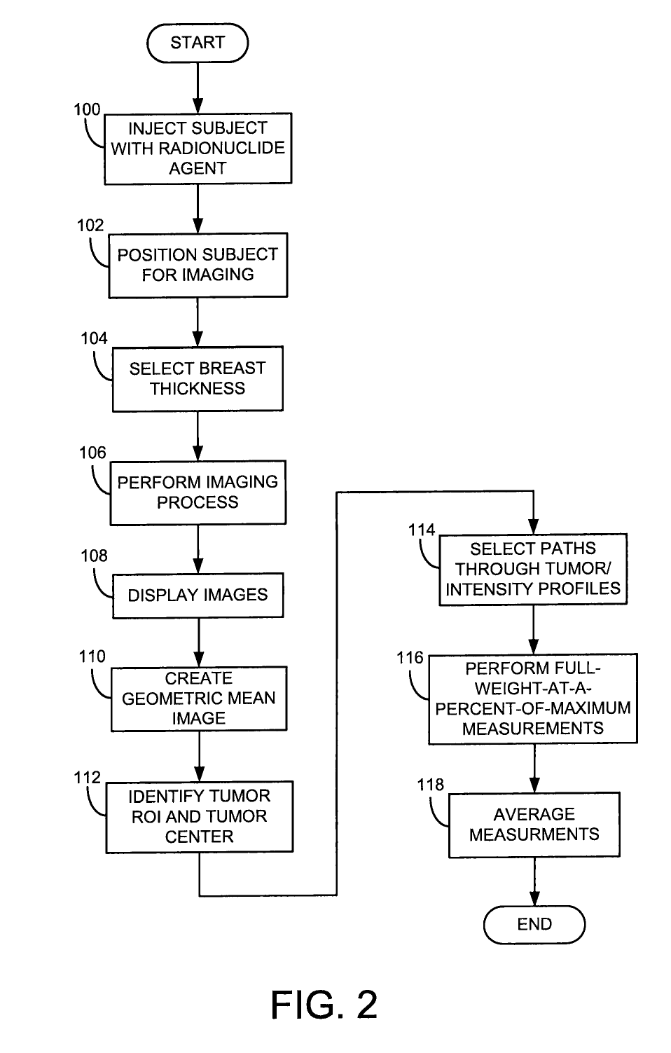

Referring to FIGS. 1 and 2, an exemplary process for imaging breast tissue begins at process block 100 by injecting a subject with a radionuclide imaging agent such as Tc-99m sestamibi (e.g., 20 mCi/injection). The subject is then positioned for imaging at process block 102. Specifically, the subject is positioned so that a breast is arranged between the detector heads 12. The detector heads 12 are then adjusted using the user control 16 to lightly compress the breast between the upper detector head 12U and lower detector head 12L to improve image contrast and reduce motion artifacts. The compression amount is approximately ⅓ that of conventional mammography and is typically improves contrast and reduces motion artifacts.

Once the subject is properly positioned, the breast thickness is selected at process block 104. Specifically, the breast thickness may be automatically determined based on the relative position of the upper detector head 12U and the lower detector head 12L or an operator may enter the breast thickness through a user interface, the display 20.

At approximately 5 minutes post-injection, the breast is imaged at process block 106. An image is acquired by each detector head 12U, 12L of each breast at multiple views. For example, an image may be acquired in craniocaudal (CC) and mediolateral oblique (MLO) positions for 10 minutes per view. Furthermore, it is contemplated that imaging may be performed at multiple directions using both the craniocaudal and mediolateral oblique breast views to obtain a three-dimensional estimate of tumor size.

At each view, the images are simultaneously acquired by the upper detector head 12U and the lower detector head 12L. Thus, for each breast, multiple sets of data are acquired that are processed by the processor 18 and then shown to the operator on the display 20 or other viewing locality at process block 108. At a minimum, it is contemplated that the operator visually evaluates the four images (lower CC, upper CC, lower MLO, upper MLO) acquired of each breast.

In addition to the images described above, it is contemplated that at least one additional image may be generated at process block 110 that is a geometric mean image of the two opposing images. As a lesion moves deeper in the breast or farther away from a given detector head 12U or 12L, the diameter of the lesion increases due to the isotropic nature of the emitted photons. For example, a lesion closer to the lower detector head 12L appears smaller in the image acquired by the lower detector head 12L than in the image acquired by the upper detector head 12U. The geometric mean image of the two opposing images created at process block 110 provides a consistent lesion size on which to perform a measurement of the size of an identified tumor for a given breast thickness. Therefore, within the geometric mean image, a given tumor has a contrast indicative of the tumor being positioned in the middle of the breast, at half the total compressed breast thickness.

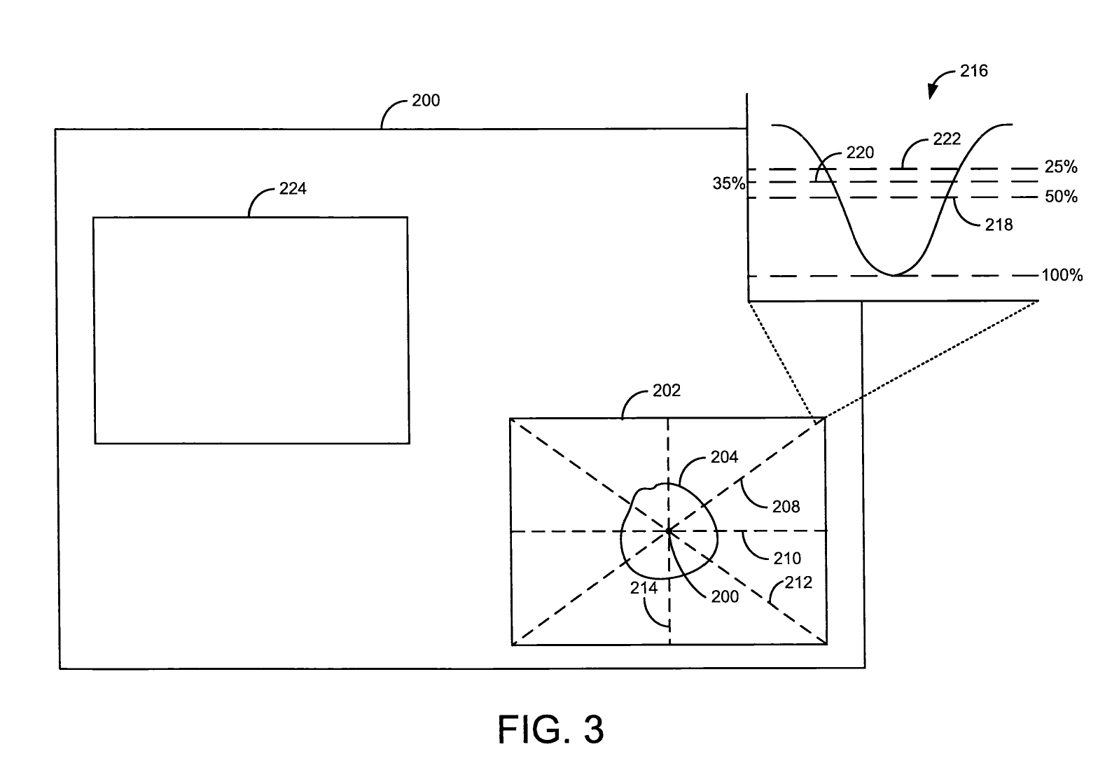

Using these images, any tumors appearing in the images are identified at process block 112 by selecting a tumor region of interest (ROI) including the tumor and indicating the center 206 of the tumor 204. For example, referring to FIG. 3, an image 200 may be displayed for an operator to select a tumor ROI 202 including evidence of a tumor 204 in the displayed image 200. Also, it is contemplated that the system may attempt to automatically identify the tumor(s) 204 within a given image or images 200 and select a preliminary ROI 202.

Referring now to FIGS. 2 and 3, with this information entered, a plurality of paths 208-214 that extend through the tumor locations/centers 204/206 are selected at process block 114. In accordance with one embodiment, at least four paths at 0, 45, 90, and −45 degrees are obtained. However, the accuracy of the size measurement can be improved by using a larger number of paths through the tumor 202 and corresponding intensity profiles. That is, as illustrated in FIG. 3, these paths 208-214 have corresponding intensity profiles 216. For each intensity profile 216, a number of full-width-at-a-percentage-of-maximum measurements 218-22 are performed at process block 116. In particular, full widths of each profile at a variety of percentages of the maximum value are measured at process block 116. For example, the full width of each profile at 10, 15, 20, 25, 30, 35, 40, and 50 percent of the maximum value can measured. However, such a large sample is not typically necessary and the full widths at, for example, 25, 35, and 50 percent may be used. Regardless of the specific number of measurements obtained, the measurements are averaged at process block 118 to provide an average measurement metric indicating the diameter/size of the identified tumor 204.

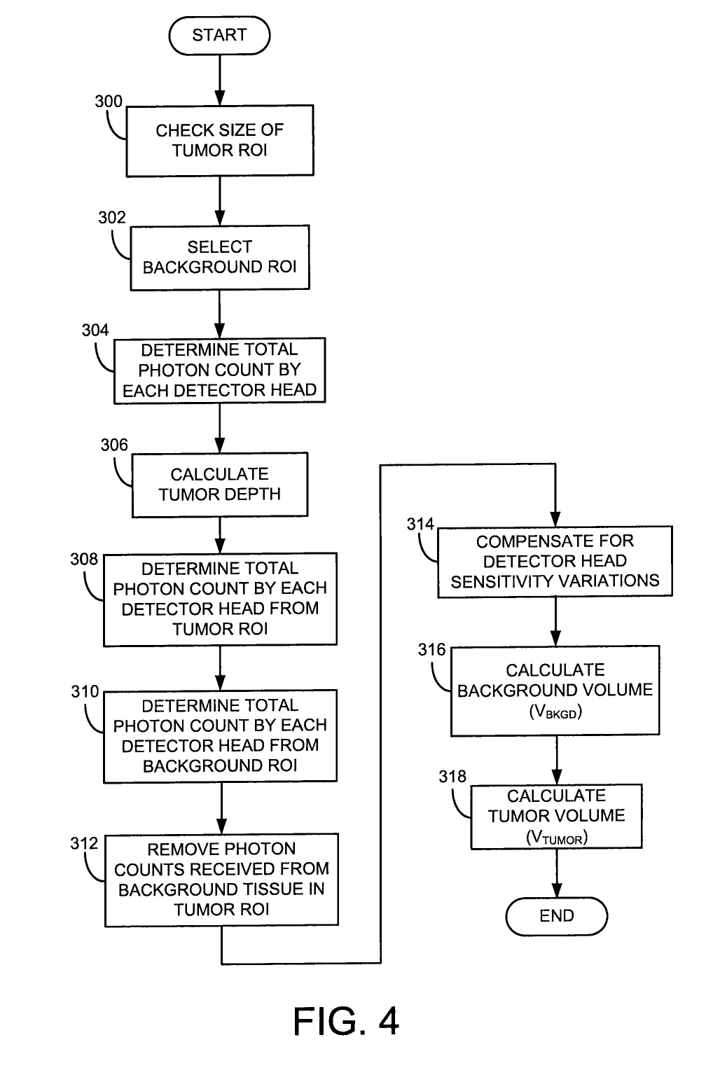

Continuing with respect to FIGS. 1 and 4, the present method described with respect to FIG. 2 can be expanded to determine the depth of an identified tumor with respect to the lower (or upper) collimator face. The method begins at process block 300 by checking the size of the tumor ROI 202 selected at process block 112 of FIG. 2. Specifically, the ROI size applied to each tumor can be large enough to include nearly all of the photon counts received from the tumor 204 by both the upper and lower detector heads and to yield the corresponding images.

To facilitate more precise placement of tumor ROIs, images can be interpolated by factors of 10 using a linear interpolation algorithm to resample the images with an adjusted pixel size, for example, 0.16×0.16 mm2. The linear algorithm calculates the resampled pixel intensities by examining the neighboring intensities of the original image and integrating them based on their proportional distance from the projected resampling position.

Referring again to FIGS. 3 and 4, once an appropriate size of the tumor ROI 202 has been confirmed, a background ROI 224 is selected at process block 302. Specifically, the reference or background ROI 224 is selected to have the same size dimensions of the tumor ROI 202, but include only background tissue that is substantially free of tumor(s).

Once the tumor and background ROIs 202, 224 have been selected, at process block 304, the number of photons received at each detector head during the imaging process is determined. The photon counts made by the lower detector head 12L and upper detector head 12U are represented as NL and NU, respectively, as follows:

N_{L}=N_{O}·exp(−μd) Eqn. 1;

N_{U}=N_{O}·exp(−μ(t−d)) Eqn. 2;

where No is the number of unattenuated photons determined at process block 304, μ is a known attenuation coefficient of soft tissue (0.153 cm-1), t is compressed breast thickness determined at process block 104 of FIG. 2, and d is tumor depth to be determined. Using these photon counts, a tumor depth calculation is performed at process block 306 by solving for d in Eqns. 1 and 2. Specifically, Eqns. 1 and 2 are solved for No and then set equal to each other to yield the following equation for tumor depth, d:

$$d = \frac{μ t - ln ( \frac{{N}_{L}}{{N}_{U}} )}{2 μ} . \qquad Eqn 3$$

Thereafter, a further refined depth measurement can be provided by removing photon counts provided by background structures in the ROI. Specifically, the sum of photon counts received from the tumor ROI identified at process block 212 of FIG. 2 and confirmed at process block 300 of FIG. 4 is calculated for each detector head at process block 308. Thereafter, at process block 310, the sum of photon counts received from the background ROI is calculated for each detector head at process block 310. With this additional information, Eqn. 3 can be modified to account for photon counts only coming from the tumor. Specifically, at process block 312, the total background photon counts received from the background ROI is subtracted from the total photon counts received from the tumor ROI to remove the photon counts from the tumor ROI that are attributable to background tissue. Also, at process block 314, a correction can be applied to the photon counts from upper detector head 12U (or, alternatively, to the lower detector head 12L if the upper detector head 12U is used as the reference frame from which the depth measurement is made) to adjust for possible differences in detector sensitivity. The steps taken at process blocks 312 and 314 are achieved by modifying Eqn. 3 yield the following:

$$d = \frac{μ t - ln ( \frac{{T}_{L} - {B}_{L}}{( {T}_{U} - {B}_{U} ) \cdot \frac{{B}_{L}}{{B}_{U}}} )}{2 μ} ; \qquad Eqn . 4$$

where TL and TU are the sum of photon counts received from an identical ROI placed on the tumor in the images provided by the lower detector head 12L and upper detector head 12U, respectively, and BL and BU are the sum of photon counts received from an ROI of equal size placed in a uniform background breast tissue region of the image provided by the lower detector head 12L and upper detector head 12U, respectively.

Additionally, using the ROI size determined as described above, photon counts in the tumor and background ROIs can be used to calculate a tumor to background (T/B) uptake ratio. To do so, the process continues by calculating a background volume (Vbkgd) at process block 316. Specifically the area of the background ROI selected at process block 302 is multiplied by the thickness of the breast determined at process block 104 of FIG. 2 to yield the background volume. Then, a tumor volume (Vtumor) is calculated at process block 318 using the tumor size/diameter calculated as described above with respect to FIG. 2. The T/B ration is therefore calculated as follows:

$$T / B Ratio = \frac{\sqrt{( {T}_{L} \cdot {T}_{U}} - \sqrt{( {B}_{L} \cdot {B}_{U}}}{\sqrt{( {B}_{L} \cdot {B}_{U}}} \cdot \frac{{V}_{Bkgd}}{{( {V}_{Tumor} )}^{F}} ; \qquad Eqn . 5$$

where F is a constant of 0.99 that was empirically determined to provide a more accurate measure of T/B ratio. In accordance with one implementation, it is contemplated that the tumor volume may be estimated by assuming a spherical tumor shape using the tumor diameter determined as described above with respect to FIG. 2. However, as described above, to more accurately determine the volume of non-spherical lesions, a higher number of intensity profiles extending in multiple directions through the tumor, using both the craniocaudal and mediolateral oblique breast views, could be used to obtain a better estimate of tumor size. In any case, Eqn. 5 is the ratio of the geometric mean of the tumor regions to the geometric mean of the background regions with corrections for differences in the ROI volumes.

It is noted that one advantage of the present implementation is that the specific pixel size used results in statistically insignificant changes in the measured diameter, depth, and T/B ratio. However, both the depth and the T/B ratio measurements are dependent on an accurate measurement of tumor size/diameter. While Eqn. 4 does not directly depend on the size/diameter measurement, the ROI size used to perform the depth measurement is determined from the previously measured tumor size. Also, as described above with respect to Eqn. 5, the T/B ratio is directly dependent on tumor volume, which is calculated using the measured tumor size/diameter.

To quantify the dependence of depth and T/B ratio calculations on the size/diameter measurement, calculations for depth and T/B ratio were performed on a set of Monte-Carlo simulated, dual-head images after setting the tumor diameter to 1 mm greater and 1 mm less than the known true diameter for each tumor. These images were acquired with a 6 cm breast thickness, a tumor depth of 2 cm from the lower detector, and a T/B of 40:1. The expected change in T/B measurement was calculated by manipulating Eqn. 5 as follows:

$$T / B Ratio ∝ \frac{1}{{( {V}_{Tumor} )}^{F}} = \frac{1}{{[ \frac{4}{3} {π ( \frac{d}{2} )}^{3} ]}^{.99}} ; \qquad Eqn . 6$$

which shows that T/B ratio is inversely proportional to the tumor volume raised to the factor F=0.99. Therefore, T/B ratio is inversely proportional to diameter, d, cubed and raised to the power of 0.99 as shown in Eqn. 6. The change in T/B ratio for a given fraction of the true diameter can thus be calculated as follows:

$$Change in T / B Ratio = \frac{1}{{( {k}^{3} )}^{.99}} ; \qquad Eqn . 7$$

where k is the fraction of the true diameter. For example, if tumor diameter is underestimated by 5%, k=0.95 and the change in T/B is 1.165, or T/B is overestimated by 16.5%. Nevertheless, the percent error in the calculated T/B ratio was tested to show the absolute average error in T/B ratio can be controlled to be less than 5% for all breast thicknesses, except at the T/B of 10:1, where error was nearly 9% at a 4 cm breast thickness.

Therefore, while depth measurements are nearly unchanged for small errors in the diameter measurement, T/B ratio can be significantly affected by a large percent error in the diameter measurement. However, as noted above, the accuracy of the diameter measurement can be improved by using more than a larger number of intensity profiles through the tumor.

MBI and MPI

In one implementation, a radiopharmaceutical is utilized as an agent to detect two different tissue abnormalities, such as myocardial abnormalities and/or breast cancer. For example, a patient is administered at least one injection of radiopharmaceuticals and may undergo both MBI and MPI within close temporal proximity of one another.

A patient undergoing MPI may receive an injection of Tc-99m sestamibi or Tc-99mm tetrofosmin both at rest and after undergoing a myocardial stress test. The patient may receive an injection of 8-10 mCi at rest and an injection of 32-40 mCi after the stress test, with total injected activities of 40-50 mCi. Patients typically wait approximately 40 minutes after the rest injection and 30 minutes after the stress injection in order to allow clearance of the radiopharmaceutical from the liver, as liver activity can interfere with imaging of the myocardium.

The MBI process can accommodate for an injection dosage that is higher than 20 mCi Tc-99m sestamibi or Tc-99m tetrofosmin, such as the dosages used during MPI. By recognizing that MBI is not impacted by uptake of these radiopharmaceuticals in other organs, such as the myocardium or the liver, the present invention permits breast imaging to commence shortly after the injection of the radiopharmaceutical, such as 5 minutes post-injection.

The duration of an MBI study may vary based on a dosage level of the radiopharmaceutical. For example, at a dosage level of 4 mCi Tc-99m sestamibi or Tc-99m tetrofosmin, the MBI studies may take approximately 40 minutes and acquire, for example, four images taking about 10 minutes each. However, at a higher administered dose of the radiopharmaceutical, such as the doses administered during MPI, the total MBI imaging time may be reduced to below 10 minutes, such as about eight minutes, still taking four 4 images, but at a rate about of two minutes per image or even down to four minutes, still taking four images, but at a rate of one image per minute.

Therefore, a patient may undergo both the nuclear cardiology procedure, such as MPI and a nuclear breast tissue imaging procedure, such as MBI, within temporal proximity of one another with no additional radiation burden or extension of examination time as compared to conducting the nuclear cardiology procedure alone. In one implementation, short imaging times for the MBI allow for the MBI to be completed during the normal wait period between injection and cardiac imaging for the cardiac studies. For example, after a resting injection, the patient is usually required to wait 40 minutes prior to commencement of the cardiac imaging. Similarly, after the stress injection, the patient, usually waits 30 minutes before the stress myocardial perfusion scan can be performed. In those time periods, a complete MBI study is performed.

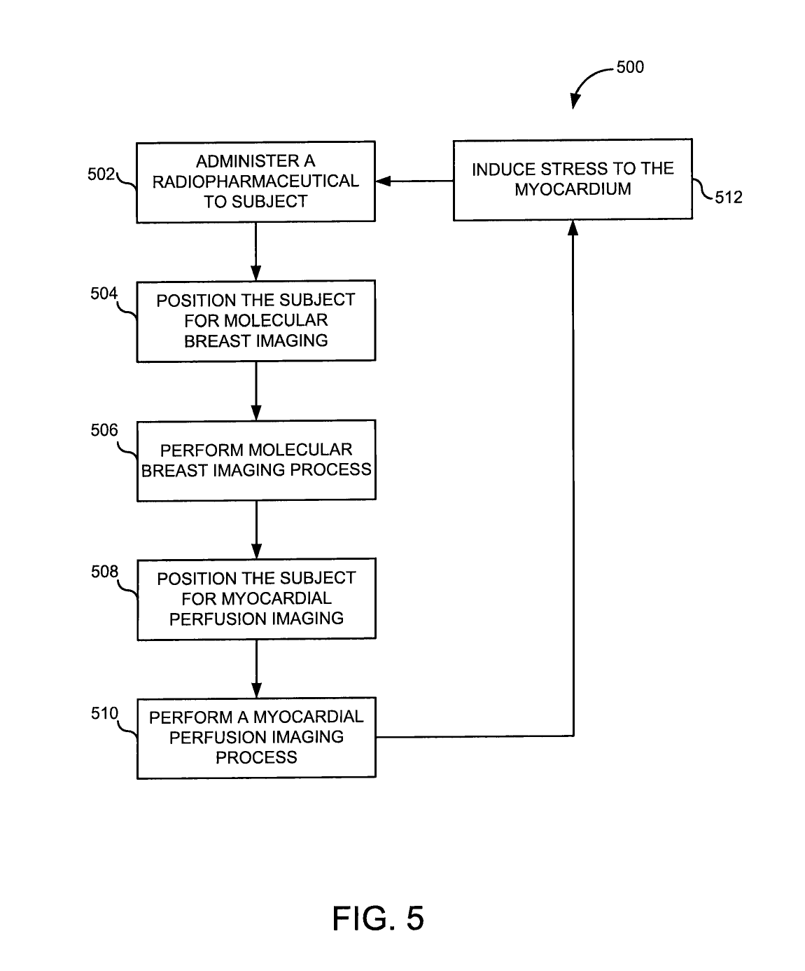

Referring to FIG. 5, an exemplary process 500 for imaging of both myocardial and breast tissue within temporal proximity of one another, begins at a process block 502. At the process block 502, the subject to be imaged is administered an injection of the radiopharmaceutical, such as 8-10 mCi of sestamibi or Tc-99mm tetrofosmin. At a process block 504, the subject is positioned for breast tissue imaging. For example, in the MBI, the patient may be seated with the breast tissue positioned between the two gamma detectors. Light compression may be applied both to reduce breast thickness and to limit movement artifact. The amount of compression applied can be manipulated based on the comfort level of the patient. Imaging can commence approximately five minutes after completion of the injection of Tc-99m sestamibi or Tc-99m tetrofosmin. At a process block 506, a breast imaging process is performed. For example, the process block 100 through the process block 118 of FIG. 2 can be performed. Similarly, the process block 300 to the process block 318 may be performed. In this manner, at least one image may be acquired of at least one breast, for example, in cranio-caudal or mediolateral oblique projections.

Referring back to FIG. 5, the subject may also undergo nuclear myocardial imaging. At a process block 508, the subject is positioned for nuclear myocardial imaging. For example, if the MPI study is conducted using a single photon emission computed tomography (SPECT) system or a Positron Emission Tomography (PET) imaging system, the patient may be positioned on an imaging table. At a process block 510, a myocardial imaging process is performed, such as using the SPECT or the PET to obtain images of the myocardium.

Optionally, if the MPI is being conducted at stress as well, the exemplary process 500 moves to a process block 512 and stress is induced to the myocardium. This can be done by exercise, such as having the subject run on a treadmill, or by administering pharmaceuticals. Pharmaceutical stress inducers include a vasodilator stress agent, such as Adenosine, or an Inotropic adrenergic agent, such as dobutamine. Process block 502 through process block 510 may optionally be repeated to obtain other breast images and/or myocardial images.

If MBI is performed after a rest injection, the image acquisition time may be in the range of approximately minutes per image, whereas after a stress injection, the breast image acquisition time may be in the range of one to three minutes per breast image. The breast images can be interpreted by a breast radiologist in a similar manner to conventional screening mammograms.

The process blocks in the exemplary process 500 may be performed in any order and some process blocks need not be performed at all. For example, if the breast tissue imaging is being performed after myocardial imaging, process block 504 and process block 506 may be performed after process block 508. Moreover, if the MBI is performed after the patient has undergone the injection of the radiopharmaceutical for the stress test, such as after the process block 512, the exemplary process 500 may begin at process block 502 and proceed to each of the following process blocks consecutively: 508, 510, 512, 502, 504, 506, 508, and 510, for example.

In another implementation, multiple breast tissue images may be taken during the stress and rest conditions. Breast tumors may demonstrate an increased blood flow under stress conditions as compared to rest conditions and, hence, can have higher tumor to background uptake after the stress injection than after the rest injection. Most benign tissues do not demonstrate this phenomenon. Therefore, the patient that has a positive MBI after the rest injection, may undergo a second MBI scan after the stress condition in the process block 512. The multiple breast images may be compared in order to obtain more diagnostic information as to the behavior of the abnormal region on the breast tissue images. For example, a computer software may compare the imaging data.

The various steps, process blocks, or acts in a method or process may be performed in the order shown, or may be performed in another order. Additionally, one or more process or method steps may be omitted or one or more process or method steps may be added to the methods and processes. An additional step, block, or action may be added in the beginning, end, or intervening existing elements of the methods and processes. Based on the disclosure and teachings provided herein, a person of ordinary skill in the art will appreciate other ways and/or methods for various implements.

It is understood that the examples and implementations described herein are for illustrative purposes only and that various modifications or changes in light thereof will be suggested to persons skilled in the art and are to be included within the spirit and purview of this application and scope of the appended claim.

1. A method for imaging a subject, the method comprising the steps of:
a) injecting the subject with a single dose of a radionuclide;
b) acquiring, with a molecular breast imaging (MBI) system, a first set of medical image data of a breast of the subject after injection of the single dose of a radionuclide;
c) acquiring, with a myocardial perfusion imaging (MPI) system, a second set of medical image data of a portion of a cardiovascular system of the subject after the single dose of a radionuclide; and
d) reconstructing the first set of medical image data into a medical image of the breast of the subject and reconstructing the second set of medical image data into a medical image of the portion of the cardiovascular system of the subject.

2. The method of claim 1 further comprising:
e) injecting the subject with a second single dose of a radionuclide;
f) acquiring, with the MBI system, a third set of medical image data of a breast of the subject after injection of the second single dose of a radionuclide;
g) acquiring, with the MPI system, a fourth set of medical image data of a portion of a cardiovascular system of the subject after the second single dose of a radionuclide; and
h) reconstructing the third set of medical image data into a second medical image of the breast of the subject and reconstructing the fourth set of medical image data into a second medical image of the portion of the cardiovascular system of the subject.

3. The method of claim 2 wherein steps a) through d) are performed after a period of rest by the subject and steps e through h) are performed at least one of during and after a period of stress by the subject.

4. The method of claim 3 further comprising registering the medical image of the breast of the subject and the second medical image of the breast of the subject and providing an indication of a change in a ratio of tumor and background structures present in the medical image of the breast of the subject and the second medical image of the breast.

5. The method of claim 1 wherein step b) includes a delay following step a) of approximately 5 minutes.

6. The method of claim 1 wherein step c) is performed at least 30 minutes after step a).

7. The method of claim 1 wherein step b) is performed before step c).

8. The method of claim 1 wherein the radionuclide is at least one of Tc-99m sestamibi and Tc-99m tetrofosmin.

9. The method of claim 1 wherein step b) is performed in less than 20 minutes.
